(19) United States
(12) Patent Application Publication
(10) Pub. No.: US 2011/0178044 A1
(43) Pub. Date: Jul. 21, 2011

(54) TRANSDERMAL ADMINISTRATION OF HYDROPHILIC DRUGS USING PERMEATION ENHANCER COMPOSITION

(76) Inventors: Tsung-Min Hsu, San Diego, CA (US); Eric Jacobson, San Diego, CA (US); Eric C. Luo, Plano, TX (US); Alan T.J. Hickey, San Diego, CA (US); Nicole T. Gricenko, San Diego, CA (US)

(21) Appl. No.: 13/079,267

(22) Filed: Apr. 4, 2011

Related U.S. Application Data
(63) Continuation of application No. 10/966,836, filed on Oct. 14, 2004, now abandoned.
(60) Provisional application No. 60/511,171, filed on Oct. 14, 2003.

(51) Int. Cl. A61K 31/663; A61K 47/02; A61K 31/5415; A61K 31/196; A61P 19/10; A61P 29/00; A61P 35/00; A61P 25/28; A61P 25/04; A61P 31/00; A61P 3/00

(52) U.S. Cl. 514/108; 514/769; 514/226.5; 514/567

(57) ABSTRACT

Methods, formulations, and drug delivery systems are provided for enhancing the flux of a transdermally administered hydrophilic drug using a basic permeation enhancer composition. The enhancer composition contains an inorganic hydroxide and a weaker, nitrogenous base, wherein the bases are selected such that a 0.1M aqueous solution of the nitrogenous base has a pH that is about 1.0 to about 6.5 lower than the pH of a 0.1M aqueous solution of the inorganic hydroxide. Additionally, the molar ratio of the nitrogenous base to the inorganic hydroxide in the enhancer composition is in the range of about 0.5n:1 to about 20n:1, where n is the number of hydroxide ions per molecule of the inorganic hydroxide.

### CROSS-REFERENCE TO RELATED APPLICATIONS

This application claims priority under 35 U.S.C. §119(e)(1) to provisional U.S. Patent Application Ser. No. 60/511,171, filed Oct. 14, 2003.

### FIELD OF THE INVENTION

This invention relates generally to the topical and transdermal administration of pharmacologically active agents, and more particularly relates to methods and compositions for enhancing the flux of a hydrophilic drug through a body surface using a basic permeation enhancer composition.

### BACKGROUND OF THE INVENTION

The delivery of drugs through the skin provides many advantages; primarily, such a means of delivery is a comfortable, convenient, and noninvasive way of administering drugs. The variable rates of absorption and metabolism encountered in oral treatment are avoided, and other inherent inconveniences, e.g., gastrointestinal irritation and the like, are eliminated as well. Transdermal drug delivery also makes possible a high degree of control over blood concentrations of any particular drug.

Skin is a structurally complex, relatively thick membrane. Molecules moving from the environment into and through intact skin must first penetrate the stratum corneum and any material on its surface. They must then penetrate the viable epidermis, the papillary dermis, and the capillary walls into the blood stream or lymph channels. To be so absorbed, molecules must overcome a different resistance to penetration in each type of tissue. Transport across the skin membrane is thus a complex phenomenon. However, it is the cells of the stratum corneum, which present the primary barrier to absorption of topical compositions or transdermally administered drugs. The stratum corneum is a thin layer of dense, highly keratinized cells approximately 10-15 microns thick over most of the body. It is believed to be the high degree of keratinization within these cells as well as their dense packing which creates in most cases a substantially impermeable barrier to drug penetration. With many drugs, the rate of permeation through the skin is extremely low without the use of some means to enhance the permeability of the skin.

Numerous chemical agents have been studied as a means of increasing the rate at which a drug penetrates through the skin. As will be appreciated by those in the field, chemical enhancers are compounds that are administered along with the drug (or in some cases the skin may be pretreated with a chemical enhancer) in order to increase the permeability of the stratum corneum, and thereby provide for enhanced penetration of the drug through the skin. Ideally, such chemical penetration enhancers or “permeation enhancers,” as the compounds are referred to herein, are compounds that are innocuous and serve merely to facilitate diffusion of the drug through the stratum corneum. The permeability of many therapeutic agents with diverse physicochemical characteristics may be enhanced using these chemical enhancement means. However, there are skin irritation and sensitization problems associated with high levels of certain enhancers.

### SUMMARY OF THE INVENTION

In U.S. Pat. No. 6,586,000 to Luo, U.S. Pat. No. 6,558,695 to Luo, U.S. Pat. No. 6,565,879 to Luo, International Patent Publication No. WO 01/43775, all of common assignment herewith to Dermatrends, Inc. (San Diego, Calif.), basic permeation enhancers such as inorganic hydroxides have been disclosed as surprisingly effective in increasing the rate at which even high molecular weight drugs permeate into and through the skin without resulting in skin damage or systemic toxicity. The present invention is directed to an improved such method in which the rate of permeation of a transdermally administered hydrophilic drug is increased significantly relative to the rate enhancement provided by use of an inorganic hydroxide alone, particularly for water-soluble, acidic drugs. The invention does not require a higher concentration of the inorganic hydroxide in the drug-containing transdermal formulation or “patch,” but rather involves the use of a basic permeation enhancer composition in which an inorganic hydroxide is combined with a weaker base.

Accordingly, in a first aspect of the invention, a method is provided for enhancing the flux of a hydrophilic drug through the body surface, i.e., the rate at which the drug permeates through an individual's skin or mucosal tissue. The method involves: (a) administering a therapeutically effective amount of the drug to a localized region of a human patient's body surface; and (b) applying an effective flux-enhancing amount of a basic permeation enhancer composition to the localized region, wherein the enhancer composition is comprised of (i) an inorganic hydroxide and (ii) a weaker, nitrogenous base. The nitrogenous base is selected such that a 0.1M aqueous solution of the base has a pH that is about 1.0 to about 6.5 lower than the pH of a 0.1M aqueous solution of the inorganic hydroxide. Additionally, the molar ratio of the nitrogenous base to the inorganic hydroxide in the composition is in the range of about 0.5n:1 to about 20n:1, where n is the number of hydroxide ions per molecule of the inorganic hydroxide. The effective flux-enhancing amount of the composition is generally an amount sufficient to provide a pH within the range of about 8.0 to about 13.0 at the localized region of the body surface during transdermal drug administration.

Another aspect of the invention pertains to a delivery system for transdermally administering a hydrophilic drug, the system comprising:

(a) at least one drug reservoir containing (i) a therapeutically effective amount of the hydrophilic drug and (ii) an effective flux-enhancing amount of a basic permeation enhancer composition containing an inorganic hydroxide and a weaker nitrogenous base, wherein the relative strengths of the two bases and their molar ratio in the basic permeation enhancer composition are as described above;

(b) a means for maintaining the system in drug- and enhancer-transmitting relationship to the body surface so as to form a body surface-system interface; and

(c) a backing layer that serves as the outer surface of the device during use.

A further aspect of the invention relates to a pharmaceutical formulation to be applied to the body surface and provide transdermal administration of a hydrophilic drug. The formulation comprises:

(a) a therapeutically effective amount of the drug;

(b) an effective flux-enhancing amount of a basic permeation enhancer composition containing an inorganic hydroxide and a nitrogenous base, wherein the relative strengths of the two bases and the molar ratio in the basic permeation enhancer composition are as described above; and

(c) a pharmaceutically acceptable carrier suitable for transdermal drug administration. The formulation may be a gel, cream, lotion, paste, or the like.

### BRIEF DESCRIPTION OF THE DRAWINGS

FIG. 1 illustrates the cumulative amount of meloxicam that permeated through skin in vitro from meloxicam solutions containing a basic permeation enhancer composition of the invention, as described in Example 1.

FIG. 2 illustrates the cumulative amount of diclofenac sodium that permeated through skin in vitro from transdermal delivery systems formulated with basic permeation enhancer compositions of the invention, as described in Example 2.

FIG. 3 illustrates the cumulative amount of alendronate sodium that permeated through skin in vitro from alendronate sodium solutions containing a basic permeation enhancer composition of the invention, as described in Example 3.

### DETAILED DESCRIPTION OF THE INVENTION

### Nomenclature, Definitions, and Overview

It is to be understood that unless otherwise indicated, this invention is not limited to specific drugs, formulation components, delivery systems, carriers, or the like, as such may vary. It is also to be understood that the terminology used herein is for the purpose of describing particular embodiments only, and is not intended to be limiting. In addition, as used in this specification and the appended claims, the singular forms “a,” “an,” and “the” include plural referents unless the context clearly dictates otherwise. Thus, for example, reference to “a hydrophilic drug” includes not only a single hydrophilic drug but also two or more hydrophilic drugs that may or may not be admixed, reference to “an inorganic hydroxide” includes a single inorganic hydroxide as well as two or inorganic hydroxides, and the like.

In describing and claiming the present invention, the following terminology will be used in accordance with the definitions set out below.

The terms “drug,” “active agent,” and “pharmacologically active agent” are used interchangeably herein to refer to any agent that is capable of producing a beneficial biological effect, preferably a pharmacological response, which may be therapeutic, diagnostic, or prophylactic in nature. The terms also encompass pharmaceutically acceptable, pharmacologically active derivatives of those drugs specifically mentioned herein, including, but not limited to, salts, esters, amides, prodrugs, active metabolites, isomers, fragments, analogs, and the like. When the terms drug,” “active agent,” and “pharmacologically active agent” are used, then, or when a particular drug is specifically identified, it is to be understood that pharmaceutically acceptable, pharmacologically active salts, esters, amides, prodrugs, active metabolites, isomers, fragments, analogs, etc. of the active agent are intended as well as the active agent per se. It should be noted that according to the present invention, a single drug may be administered or two or more drugs may be administered in combination.

By a “therapeutically effective amount” of a drug is meant a nontoxic but sufficient amount of the drug to provide the desired beneficial effect. For example, a therapeutically effective amount of a drug intended to treat an individual afflicted with a disorder, disease, or other adverse physiological condition is an amount that will effect a reduction in severity and/or frequency of symptoms, eliminate the symptoms and/or their underlying cause, and/or facilitate improvement or remediation of damage. As another example, a therapeutically effective amount of a drug given to a clinically asymptomatic individual who is susceptible to a particular disorder, disease, or other adverse physiological condition, is an amount that will prevent the occurrence of symptoms and/or their underlying cause.

The amount of drug that is “therapeutically effective” will vary from subject to subject, depending on the age and general condition of the individual, the particular drug or drugs, and the like. Thus, it is not always possible to specify an exact “effective amount.” However, an appropriate “effective” amount in any individual case may be determined by one of ordinary skill in the art using routine experimentation. When the term “therapeutic effective amount” is used to refer to an amount in a formulation that is not in a finite dosage form, e.g., a formulation that is a gel, cream, lotion, paste, or the like, the term refers to a concentration of the drug in the formulation that corresponds to a therapeutically effective amount of the drug in a unit dosage of the formulation.

By “pharmaceutically acceptable,” such as in the recitation of a “pharmaceutically acceptable carrier” or a “pharmaceutically acceptable additive,” is meant a material that is not biologically or otherwise undesirable, i.e., the material may be incorporated into a pharmaceutical formulation or delivery system of the invention without causing any appreciable biological effects or interacting in a deleterious manner with any of the other components of the formulation or system in which it is contained. “Pharmacologically active,” as in a “pharmacologically active” derivative of a drug, refers to a derivative having the same type of pharmacological activity as the parent compound and approximately equivalent in degree. When the term “pharmaceutically acceptable” is used to refer to a derivative (e.g., a salt) of an active agent, it is to be understood that the compound is pharmacologically active as well. When the term “pharmaceutically acceptable” is used to refer to a carrier or excipient, it implies that the excipient has met the standards of toxicological and manufacturing testing required by the U.S. Food & Drug Administration for inactive ingredients.

The term “aqueous” as applied to a formulation of the invention is used to indicate that the formulation contains water or becomes water-containing following application to the skin or mucosal tissue.

“Penetration enhancement” or “permeation enhancement” as used herein relates to an increase in the permeability of the skin or mucosal tissue to the selected pharmacologically active agent so that the rate at which the agent permeates therethrough, i.e., the “flux” of the agent through the body surface, is increased relative to the rate that would be obtained in the absence of permeation enhancer. The enhanced permeation effected through the use of such enhancers can be observed by measuring the rate of diffusion of drug through animal or human skin using, for example a Franz diffusion apparatus as known in the art and as employed in the Examples herein.

An “effective permeation enhancing amount” of a permeation enhancer composition of the invention refers to a nontoxic, non-damaging but sufficient amount of the enhancer composition to provide the desired increase in flux of a drug through human skin or mucosal tissue, and, correspondingly, the desired depth of penetration, rate of administration, and amount of drug delivered.

A “localized region” of skin or mucosal tissue refers to the area of an individual's body surface through which a drug-enhancer formulation is delivered, and is a defined area of intact unbroken living skin or mucosal tissue. That area will usually be in the range of about 5-200 cm², more usually in the range of about 5-100 cm², preferably in the range of about 20-60 cm². However, it will be appreciated by those skilled in the art of drug delivery that the area of skin or mucosal tissue through which drug is administered may vary significantly, depending on the nature of the formulation, the particular drug administered, the intended dose, the patch configuration, and the like.

“Transdermal” drug delivery is meant administration of a drug to the skin surface of an individual so that the drug passes through the skin tissue and into the individual's blood stream, thereby providing a systemic effect. The term “transdermal” is intended to include “transmucosal” drug administration, i.e., administration of a drug to a mucosal (e.g., sublingual, buccal, vaginal, rectal) surface within an individual's body so that the drug passes through the mucosal tissue and into the blood stream.

The invention provides for transdermal administration of a hydrophilic drug using a basic enhancer composition containing an inorganic hydroxide and a second, weaker base, such that the flux of the drug through an individual's body surface is increased relative to the flux of the drug obtained using only an inorganic hydroxide as a permeation enhancer. The invention provides for a marked increase in flux without necessitating use of a higher concentration of inorganic hydroxide, which would be likely to result in irritation or other problems for a number of patients. Surprisingly, the increase in the degree of enhancement is far higher than would be expected upon combining the two types of bases in a single formulation or delivery system. In addition, the pH of the system is maintained at an elevated level for a longer time period than possible with prior systems containing only an inorganic hydroxide as a permeation enhancer. This in turn ensures that with a hydrophilic drug whose aqueous solubility decreases with decreasing pH (typically acidic drugs), the drug will be delivered over an extended time period without precipitation.

Accordingly, the invention provides a method, delivery system, and formulation for transdermally administering a hydrophilic drug at an enhanced permeation rate for an extended time period. The method involves administering a therapeutically effective amount of the drug to a localized region of body surface and applying an effective flux-enhancing amount of a basic permeation enhancer composition to the localized region. The basic permeation enhancer composition, the hydrophilic drug, suitable delivery systems, and pharmaceutical formulations are described below.

### The Permeation Enhancer Composition

The basic permeation enhancer composition contains an admixture of an inorganic hydroxide and a nitrogenous base, wherein a 0.1M aqueous solution of the nitrogenous base has a pH that is about 1.0 to about 6.5 lower than a 0.1M aqueous solution of the inorganic hydroxide, and preferably about 1.5 to about 6.5 lower than the pH of a 0.1M aqueous solution of the inorganic hydroxide. In addition, the molar ratio of the nitrogenous base to the inorganic hydroxide in the enhancer composition is in the range of about 0.5n:1 to about 20n:1, where n is the number of hydroxide ions per molecule of the inorganic hydroxide. Thus, for ammonium hydroxide or an alkali metal hydroxide such as sodium hydroxide, n is 1 and the molar ratio of the nitrogenous base to the inorganic hydroxide is therefore in the range of about 0.5:1 to about 20:1. For an alkaline earth metal hydroxide such as calcium hydroxide, n is 2 and the molar ratio of the nitrogenous base to the inorganic hydroxide is thus in the range of about 1:1 to about 40:1. Preferably, the molar ratio of the nitrogenous base to the inorganic hydroxide in the enhancer composition is in the range of about 0.5n:1 to about 10n:1. It will be appreciated that stronger and/or higher molecular weight nitrogenous bases will be used in lesser quantities, while relatively weak and/or lower molecular weight nitrogenous bases will be used in greater quantities.

The inorganic hydroxide is generally selected from alkali metal hydroxides, alkaline earth metal hydroxides, ammonium hydroxide, and combinations thereof. Alkali metal hydroxides include sodium hydroxide and potassium hydroxide, while alkaline earth metal hydroxides include calcium hydroxide and magnesium hydroxide. Preferred inorganic hydroxides are alkali metal hydroxides, particularly sodium hydroxide. As indicated in the table below, a 0.1M aqueous solution of an alkali metal hydroxide has a pH of about 13.0 when measured at 25° C.

The other component of the basic permeation enhancer composition, i.e., the nitrogenous base, can be a primary amine, a secondary amine, a tertiary amine, an amide, an oxime, a nitrile, a nitrogen-containing heterocycle, or urea. Mixtures of nitrogenous bases can also be used.

Preferred nitrogenous bases herein are amino alcohols and urea. Exemplary amino alcohols are those of the formula NR¹R²R³ wherein R¹ is hydroxyl-substituted C₁-C₁₈ hydrocarbyl, and R² and R³ are selected from H, C₁-C₁₈ hydrocarbyl (optionally substituted with a substituent other than hydroxyl), and hydroxyl-substituted C₁-C₁₈ hydrocarbyl. Of these, preferred amino alcohols are those wherein R¹ is C₁-C₁₂ alkyl substituted with 1 to 12 hydroxyl groups, and R² and R³ are selected from H, C₁-C₁₋₁₂ alkyl (optionally substituted with substituents other than hydroxyl), and C₁-C₁₂ alkyl substituted with 1 to 12 hydroxyl groups, and more preferred amino alcohols are those wherein R¹ is C₁-C₆ alkyl substituted with 1 to 5 hydroxyl groups, and R² and R³ are selected from H, C₁-C₆ alkyl, and C₁-C₆ alkyl substituted with 1 to 5 hydroxyl groups. Specific examples of the more preferred amino alcohols, then, are triethanolamine (R¹, R², and R³ are —CH₂CH₂OH), diethanolamine (R¹ and R² are —CH₂CH₂OH, and R³ is H), N-methyl glucamine (R¹ is —CH₂—[CH(OH)]₄—CH₂OH, R² is CH₃, and R³ is H) (also referred to as “meglumine”), 2-amino-2-methyl-1,3 propanediol (R¹ is —C(CH₃)(CH₂OH)₂, and R² and R³ are H), and 2-amino-2-methyl-1-propanol (R¹ is —C(CH₃)₂(CH₂OH), and R² and R³ are H).

Other preferred nitrogenous bases include, without limitation: alkylamines (including mono-, di-, and tri-alkylamines) such as methylamine, ethylamine, isopropylamine, n-butylamine, 2-aminoheptane, cyclohexylamine, ethylenediamine, and 1,4-butanediamine; arylamines and aralkylamines such as aniline, N,N-diethylaniline, benzylamine, α-methylbenzylamine, and phenethylamine; aromatic nitrogen-containing heterocycles such as 2-amino-pyridine, benzimidazole, 2,5-diaminopyridine, 2,4-dimethylimidazole, 2,3-dimethylpyridine, 2,4-dimethylpyridine, 3,5-dimethylpyridine, imidazole, methoxypyridine, γ-picoline, and 2,4,6-trimethylpyridine; and non-aromatic nitrogen-containing heterocycles such as 1,2-dimethylpiperidine, 2,5-dimethylpiperazine, 1,2-dimethylpyrrolidine, 1-ethylpiperidine, n-methylpyrrolidine, morpholine, and piperazine.

The strengths of representative bases useful in conjunction with the invention are as follows:

| Base | pH of 0.1M aqueous sol'n | pKₐ | pK_{b} |
|---|---|---|---|
| sodium hydroxide | 13.0 | — | — |
| potassium hydroxide | 13.0 | — | — |
| triethanolamine | 10.38 | 7.78 | 6.24 |
| diethanolamine | 10.94 | 8.88 | 5.12 |
| N-methyl glucamine | 11.26 | 9.52 | 4.48 |
| urea | 6.55 | 0.10 | 13.9 |
| methylamine | 11.82 | 10.66 | 3.34 |
| ethylamine | 11.90 | 10.81 | 3.19 |
| isopropylamine | 11.80 | 10.60 | 3.40 |
| n-butylamine | 11.81 | 10.61 | 3.39 |
| 2-aminoheptane | 11.85 | 10.70 | 3.30 |
| cyclohexylamine | 11.82 | 10.64 | 3.36 |
| ethylenediamine | 11.46 | 9.93 | 4.07 |
| 1,4-butanediamine | 11.90 | 10.80 | 3.20 |
| aniline | 8.82 | 4.63 | 9.37 |
| N,N-diethylaniline | 7.81 | 2.61 | 11.39 |
| benzylamine | 11.17 | 9.33 | 4.67 |
| α-methyl-benzylamine | 11.38 | 9.75 | 4.25 |
| phenethylamine | 11.42 | 9.83 | 4.17 |
| 2-aminopyridine | 9.91 | 6.82 | 7.18 |
| benzimidazole | 9.24 | 5.48 | 8.52 |
| 2,5-diaminopyridine | 9.74 | 6.48 | 7.52 |
| 2,4-dimethylimidazole | 10.68 | 8.36 | 5.64 |
| 2,3-dimethylpyridine | 9.79 | 6.57 | 7.43 |
| 2,4-dimethylpyridine | 10.00 | 6.99 | 7.01 |
| 3,5-dimethylpyridine | 9.58 | 6.15 | 7.85 |
| imidazole | 9.96 | 6.92 | 7.08 |
| 2-methoxypyridine | 8.14 | 3.28 | 10.72 |
| 3-methoxypyridine | 8.89 | 4.78 | 9.22 |
| 4-methoxypyridine | 9.79 | 6.58 | 7.42 |
| γ-picoline | 9.50 | 5.99 | 8.01 |
| 2,4,6-trimethylpyridine | 9.85 | 6.69 | 7.31 |
| 1,2-dimethylpiperidine | 11.61 | 10.22 | 3.78 |
| 2,5-dimethylpiperazine | 11.33 | 9.66 | 4.34 |
| 1,2-dimethylpyrrolidine | 11.60 | 10.20 | 3.80 |
| 1-ethylpiperidine | 11.73 | 10.45 | 3.55 |
| N-methylpyrrolidine | 11.73 | 10.46 | 3.54 |
| morpholine | 10.75 | 8.50 | 5.50 |
| piperazine | 11.42 | 9.83 | 4.17 |

Preferably, the amount of the basic permeation enhancer composition applied to the localized region of the body surface in combination with the hydrophilic drug is an amount sufficient to provide a pH within the range of about 8.0 to about 13.0, preferably about 8.5 to about 11.5, more preferably about 9.5 to 11.5, and most preferably about 10.0 to about 11.5, at the localized region of the body surface during administration of the drug.

The pH at the localized region of the body surface is the pH at the interface of the body surface and a delivery system or pharmaceutical formulation of the invention. Anhydrous delivery systems and formulations may not have a measurable pH, and the system or formulation can then be designed so as to provide a target pH at the interface. In various types of delivery systems and formulations, moisture from the body surface can migrate into the system or formulation, dissolve the basic permeation enhancer composition, and thus release the basic composition into solution, which will then provide the desired target pH at the interface. In some cases, the desired pH at the localized region of body surface is provided by applying a formulation (or delivery system containing the formulation) containing the hydrophilic drug and the basic permeation enhancer composition, wherein the formulation itself has a pH in the in the range of about 8.0 to about 13.0, preferably about 8.5 to about 11.5, more preferably about 9.5 to about 11.5, and most preferably about 10.0 to about 11.5. In other cases, the body surface is exposed to the basic permeation enhancer composition for a time period sufficient to provide a high pH at the body surface, thus creating channels in the skin or mucosa for the drug to go through. It is expected that drug flux is proportional to the strength of the solution and the duration of exposure. However, it is desirable to balance the maximization of drug flux with the minimization of skin damage. This can be done in numerous ways. For example, the skin damage may be minimized by selecting a lower pH within the 8.0-13.0 range, by exposing the skin to the formulation or system for a shorter period of time, or by including at least one irritation-mitigating additive. Alternatively, the patient can be advised to change the location of application with each subsequent administration.

Depending on the selected hydrophilic drug and on the components of the basic enhancer composition, the enhancer composition will typically represent about 0.3 wt. % to about 7.0 wt. %, preferably about 0.5 wt. % to about 4.0 wt. %, more preferably about 0.5 wt. % to about 3.0 wt. %, most preferably about 0.75 wt. % to about 2.0 wt. %, of a topically applied formulation or of a drug reservoir of a drug delivery system. Greater fractions may be used by controlling the rate and/or quantity of release of the basic permeation enhancer composition, preferably during the drug delivery period.

### The Hydrophilic Drug

The drug administered using the method, delivery system, and formulation of the invention is hydrophilic. The terms “hydrophilic” and “hydrophobic” are generally defined in terms of a partition coefficient P, which is the ratio of the equilibrium concentration of a compound in an organic phase to that in an aqueous phase. A hydrophilic compound herein generally, although not necessarily, has a log P value less than 3.5, usually less than 1.0, and most typically less than about −0.5, where P is the partition coefficient of the compound between octanol and water (while, by contrast, hydrophobic compounds will generally have a log P of at least 3.5, typically greater than about 5.0). Preferred hydrophilic drugs herein are water soluble, particularly in a basic aqueous solution. Generally, hydrophilic drugs for transdermal administration according to the invention have an apparent aqueous solubility greater than 2.5 mg/ml at a pH of 8.0 when measured at 25° C., and the preferred hydrophilic drugs herein have an apparent aqueous solubility greater than 10 mg/ml at a pH of 8.0 when measured at 25° C.

For purposes of the present invention, hydrophilic drugs also include drugs that may not have a log P of less than 3.5, but do have an apparent aqueous solubility that increases with increasing pH. Preferred such drugs exhibit the above-mentioned apparent aqueous solubility profile, i.e., have an apparent aqueous solubility greater than 2.5 mg/ml at a pH of 8.0 when measured at 25° C., and more preferably have an apparent aqueous solubility greater than 10 mg/ml at a pH of 8.0 when measured at 25° C.

Also included within the scope of “hydrophilic” drugs in the present context are ionizable active agents that are generally viewed as hydrophobic when in non-ionized form, but that are hydrophilic—in terms of log P and/or apparent aqueous solubility—when ionized. That is, for example, a non-ionized ionizable drug D-COOH (where D is the molecular core of the drug and H is a hydrogen atom) that is hydrophobic when in electronically neutral, non-ionized form, may nevertheless serve as a hydrophilic drug herein when converted to the anion DCOO⁻ in association with cationic counterion, i.e., to a basic addition salt.

Within the aforementioned parameters, then, the hydrophilic drug administered may be any compound that is suitable for transdermal delivery and induces a desired local or systemic beneficial effect. Such compounds include the broad classes of compounds normally delivered through body surfaces and membranes, including skin. While appreciating the fact that drugs may be classified in more than one category, exemplary categories of interest include, without limitation, analgesic agents, anesthetic agents, anti-anginal agents, antiarthritic agents, anti-arrhythmic agents, antiasthmatic agents, antibiotic agents, anticancer agents, anticholinergic agents, anticoagulants, anticonvulsants, antidepressants, antidiabetic agents, antifungal agents, antiglaucoma agents; antigout agents, antihelminthic agents, antihistamines, antihyperlipidemic agents, antihypertensive agents, antiinflammatory agents, antimalarial agents, antimigraine agents, antimuscarinic agents, antinauseants, anti-obesity agents, anti-osteoporosis agents, antipanic agents; antiparkinsonism agents, antiprotozoal agents, antipruritics, antipsychotic agents, antipyretics, antitubercular agents, antitussive agents, antiulcer agents, antiviral agents, anxiolytics, appetite suppressants, calcium channel blockers, cardiac inotropic agents, beta-blockers, bone density regulators, central nervous system stimulants, cognition enhancers, corticosteroids, decongestants, diuretics, gastrointestinal agents, genetic materials, hormonolytics, hypnotics, hypoglycemic agents, immunosuppressants, keratolytics, leukotriene inhibitors, macrolides, mitotic inhibitors, muscle relaxants, narcotic antagonists, neuroleptic agents, nicotine, parasympatholytic agents, peptides, polypeptides, proteins, saccharides, sedatives, sex hormones, sympathomimetic agents, tocolytics, tranquilizers, vasodilators, vitamins, and combinations thereof.

In one preferred embodiment, the hydrophilic drug is an antiinflammatory agent, generally a nonsteroidal antiinflammatory agent (NSAID) or COX-2 inhibitor. Specific examples of such drugs include, without limitation, acetylsalicylic acid, alclofenac, alminoprofen, benoxaprofen, butibufen, bucloxic acid, carprofen, celecoxib, clidenac, diclofenac, diflunisal, etodolac, fenbufen, fenoprofen, fentiazic, flufenamic acid, flufenasol, flurbiprofen, furofenac, ibufenac, ibuprofen, indomethacin, indoprofen, isoxepac, isoxicam, ketoprofen, ketorolac, meclofenamic acid, mefenamic acid, meloxicam, miroprofen, naproxen, oxaprozin, oxyphenbutazone, oxpinac, parecoxib, phenylbutazone, piclamilast, piroxicam, pirprofen, pranoprofen, rofecoxib, sudoxicam, sulindac, suprofen, tenclofenac, tiaprofenic acid, tolfenamic acid, tolmetin, tramadol, valdecoxib, zomepirac, and pharmacologically active basic addition salts thereof.

In another preferred embodiment, the hydrophilic drug is a bisphosphonic acid derivative useful in the diagnosis and treatment of disorders and conditions related to bone resorption, calcium metabolism, and phosphate metabolism. Examples of these bisphosphonic acids include 1-hydroxyethane-1,1-diphosphonic acid (etidronic acid), 1,1-dichloromethylene-1,1-bisphosphonic acid (clodronic acid), 3-amino-1-hydroxypropylidene-1,1-bisphosphonic acid (pamidronic acid), 4-amino-1-hydroxybutylidene-1,1-bisphosphonic acid (alendronic acid), 6-amino-1-hydroxyhexylidene-1,1-bisphosphonic acid (neridronic acid), (4-chlorophenyl)thiomethane-1,1-diphosphonic acid (tiludronic acid), 1-hydroxy-2-(3-pyridinyl)-ethylidene-1,1-bisphosphonic acid (risedronic acid), cycloheptylaminomethylene-1,1-bisphosphonic acid (cimadronic acid), 1-hydroxy-3-(N-methyl-N-pentylamino) propylidene-1,1-bisphosphonic acid (ibandronic acid), 3-(dimethylamino)-1-hydroxypropylidene-1,1-bisphosphonic acid (olpadronic acid), [2-(2-pyridinyl)ethylidene]-1,1-bisphosphonic acid (piridronic acid) and 1-hydroxy-2-(1H-imidazol-1-yl)ethylidene-1,1-bisphosphonic acid (zoledronic acid).

Acidic drugs will generally, although not necessarily, be incorporated into the drug delivery systems and formulations of the invention in the form of a basic addition salt. Basic addition salts of acidic drugs are prepared from the free acid using conventional methodology, involving reaction with a pharmaceutically acceptable base. Such bases include, by way of example, organic bases such as ethylamine, n-butylamine, n-hexylamine, di-isopropylamine, trimethylamine, triethylamine, 2-diethylaminoethanol, lysine, and choline, and inorganic bases such as sodium hydroxide, potassium hydroxide, ammonium hydroxide, and calcium hydroxide.

Representative basic addition salts of hydrophilic drugs include, without limitation, diclofenac sodium, cromolyn sodium, ketorolac tromethamine, tolmetin sodium, meclofenamate sodium, and etidronate sodium. The basic addition salts may also be associated with water molecules and thus in the form of a hydrate; one such example is 4-amino-1-hydroxy-butylidene-1,1-bisphosphonic acid monosodium salt trihydrate, also known as “alendronate.”

Still other preferred drugs that can be advantageously administered using the methods, compositions, and systems of the invention are “biomolecules,” i.e., organic molecules (whether naturally occurring, recombinantly produced, or chemically synthesized in whole or in part) that are, were, or can be a part of a living organism. Biomolecules encompass, for example, nucleotides, amino acids, and monosaccharides, as well as oligomeric and polymeric species such as oligonucleotides and polynucleotides, peptidic molecules such as oligopeptides, polypeptides and proteins, saccharides such as disaccharides, oligosaccharides, polysaccharides, mucopolysaccharides or peptidoglycans (peptido-polysaccharides), and the like.

For those active agents that are chiral in nature and can thus be in an enantiomerically pure form or in a racemic mixture, the drug may be incorporated into a drug delivery system or formulation either as the racemate or in the enantiomerically pure form.

The amount of drug administered will depend on a number of factors and will vary from subject to subject and depend on the particular drug administered, the particular disorder or condition being treated, the severity of the symptoms, the subject's age, weight, and general condition, and the judgment of the prescribing physician. Other factors, specific to transdermal drug delivery, include the solubility and permeability of the carrier and adhesive layer in a drug delivery device, if one is used, and the period of time for which such a system will be fixed to the skin or other body surface. The minimum amount of drug is determined by the requirement that sufficient quantities of drug must be present in a device or composition to maintain the desired rate of release over the given period of application. The maximum amount for safety purposes is determined by the requirement that the quantity of drug present cannot exceed a rate of release that reaches toxic levels. Generally, the maximum concentration is determined by the amount of agent that can be received in the carrier without producing adverse histological effects such as irritation, an unacceptably high initial pulse of agent into the body, or adverse effects on the characteristics of the delivery device such as the loss of tackiness, viscosity, or deterioration of other properties.

### Pharmaceutical Formulations and Drug Delivery Systems

The method will typically involve application of a formulation or drug delivery system containing a hydrophilic drug and a basic permeation enhancer composition as described above to a predetermined area of the skin or other tissue for a period of time sufficient to provide the desired local or systemic beneficial effect. The method may involve direct application of the formulation as a gel, cream, lotion, paste, or the like, or may involve use of a drug delivery device. In either case, water is preferably present in order for the hydroxide ions to be provided by the bases, and thus enhance the flux of the hydrophilic drug through the patient's body surface. Thus, such a formulation or drug reservoir may be aqueous, i.e., contain water, or may be nonaqueous and used in combination with an occlusive backing layer so that moisture evaporating from the body surface is maintained within the formulation or transdermal system during drug administration. In some cases, however, e.g., with an occlusive gel, a nonaqueous formulation may be used with or without an occlusive backing layer.

Suitable formulations include gels, creams, lotions, pastes, and the like.

As will be appreciated by those working in the field of pharmaceutical formulation, gels are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the “carbomer” family of polymers, e.g., carboxypolyalkylenes that may be obtained commercially under the Carbopol® trademark. Also preferred are hydrophilic polymers such as polyethylene oxides, polyoxyethylene-polyoxypropylene copolymers, and polyvinylalcohol; cellulosic polymers such as hydroxypropyl cellulose, hydroxyethyl cellulose, hydroxypropyl methylcellulose, hydroxypropyl methylcellulose phthalate, and methylcellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Creams, as also well known in the art, are viscous liquids or semisolid emulsions, either oil-in-water or water-in-oil. Cream foundations are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant.

Lotions, which are preferred for delivery of cosmetic agents, are preparations to be applied to the skin surface without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like.

Pastes are semisolid dosage forms in which the active agent is suspended in a suitable foundation. Depending on the nature of the foundation, pastes are divided between fatty pastes or those made from single-phase, aqueous gels. The foundation in a fatty paste is generally petrolatum or hydrophilic petrolatum or the like. The pastes made from single-phase aqueous gels generally incorporate carboxymethylcellulose or the like as the foundation.

Formulations may also be prepared with liposomes, micelles, and microspheres. Liposomes are microscopic vesicles having a lipid wall comprising a lipid bilayer, and can be used as drug delivery systems herein as well. Generally, liposome formulations are preferred for poorly soluble or insoluble pharmaceutical agents. Liposomal preparations for use in the instant invention include cationic (positively charged), anionic (negatively charged) and neutral preparations. Cationic liposomes are readily available. For example, N-[1-2,3-dioleyloxy)propyl]-N,N,N-triethylammonium liposomes are available under the tradename Lipofectin® (GIBCO BRL, Grand Island, N.Y.). Anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline, dioleoylphosphatidyl glycerol, dioleoylphoshatidyl ethanolamine, among others. These materials can also be mixed with N-[1-2,3-dioleyloxy)propyl]-N,N,N-triethylammonium (DOTMA) in appropriate ratios. Methods for making liposomes using these materials are well known in the art.

Micelles are known in the art and are comprised of surfactant molecules arranged so that their polar headgroups form an outer spherical shell, while the hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethyl-ammonium chloride, dodecylammonium chloride, polyoxyl 8 dodecyl ether, polyoxyl 12 dodecyl ether, nonoxynol 10 and nonoxynol 30. Micelle formulations can be used in conjunction with the present invention either by incorporation into the reservoir of a topical or transdermal delivery system, or into a formulation to be applied to the body surface.

Microspheres, similarly, may be incorporated into the present formulations and drug delivery systems. Like liposomes and micelles, microspheres essentially encapsulate a drug or drug-containing formulation. They are generally, although not necessarily, formed from lipids, preferably charged lipids such as phospholipids. Preparation of lipidic microspheres is well known in the art and described in the pertinent texts and literature.

Various additives, known to those skilled in the art, may be included in the topical formulations. For example, solvents, including relatively small amounts of alcohol, may be used facilitate the solubilization of certain drugs. Other optional additives include opacifiers, antioxidants, fragrance, colorant, gelling agents, thickening agents, stabilizers, surfactants and the like. Other agents may also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof.

The formulation may also contain irritation-mitigating additives to minimize or eliminate the possibility of skin irritation or skin damage resulting from the drug, the base enhancer, or other components of the formulation. Suitable irritation-mitigating additives include, for example: α-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. The irritation-mitigating additive, if present, will be incorporated into the formulation at a concentration effective to mitigate irritation or skin damage, typically representing not more than about 20 wt. %, more typically not more than about 5 wt. %, of the formulation.

An alternative and preferred method involves the use of a drug delivery system, e.g., a topical or transdermal “patch,” wherein the active agent is contained within a laminated structure that is to be affixed to the skin. In such a structure, the drug composition is contained in a layer, or “reservoir,” underlying an upper backing layer that serves as the outer surface of the device during use. The laminated structure may contain a single reservoir, or it may contain multiple reservoirs.

Accordingly, another embodiment of the invention is a delivery system for transdermally administering a hydrophilic drug, the system comprising:

(a) at least one drug reservoir containing (i) a therapeutically effective amount of the hydrophilic drug and (ii) an effective flux-enhancing amount of a basic permeation enhancer composition containing an inorganic hydroxide and a nitrogenous base, wherein the relative strengths of the two bases and the molar ratio in the basic permeation enhancer composition are as described above;

(b) a means for maintaining the system in drug- and enhancer-transmitting relationship to the body surface so as to form a body surface-system interface; and

(c) a backing layer that serves as the outer surface of the device during use.

The drug reservoir of the system may comprise a polymeric matrix of a pharmaceutically acceptable adhesive material that serves to affix the system to the skin during drug delivery; typically, the adhesive material is a pressure-sensitive adhesive (PSA) that is suitable for long-term skin contact, and which should be physically and chemically compatible with the active agent, inorganic or organic base, and any carriers, vehicles or other additives that are present. Examples of suitable adhesive materials include, but are not limited to, the following: polyethylenes; polysiloxanes; polyisobutylenes; polyacrylates; polyacrylamides; polyurethanes; plasticized ethylene-vinyl acetate copolymers; and tacky rubbers such as polyisobutene, polybutadiene, polystyrene-isoprene copolymers, polystyrene-butadiene copolymers, and neoprene (polychloroprene). Preferred adhesives are polyisobutylenes, and particularly preferred adhesives are comprised of polyisobutylene and a crosslinked poly(N-vinyl lactam) such as crosslinked poly(vinyl pyrrolidone) (“Crospovidone”). Transdermal delivery systems in which a single matrix layer serves as both the drug reservoir and the skin contact adhesive are generally referred to as “monolithic” delivery systems.

The backing layer functions as the primary structural element of the transdermal delivery device and provides flexibility and, preferably, occlusivity. The material used for the backing layer should be inert and incapable of absorbing the drug, the basic permeation enhancer composition, or other components of the formulation contained within the device. The backing is preferably comprised of a flexible elastomeric material that serves as a protective covering to prevent loss of drug and/or vehicle via transmission through the upper surface of the patch, and will preferably impart a degree of occlusivity to the system, such that the area of the body surface covered by the patch becomes hydrated during use. The material used for the backing layer should permit the device to follow the contours of the skin and be worn comfortably on areas of skin such as at joints or other points of flexure, that are normally subjected to mechanical strain with little or no likelihood of the device disengaging from the skin due to differences in the flexibility or resiliency of the skin and the device. The materials used as the backing layer are either occlusive or permeable, as noted above, although occlusive backings are preferred, and are generally derived from synthetic polymers (e.g., polyester, polyethylene, polypropylene, polyurethane, polyvinylidine chloride, and polyether amide), natural polymers (e.g., cellulosic materials), or macroporous woven and nonwoven materials.

During storage and prior to use, the laminated structure preferably includes a release liner. Immediately prior to use, this layer is removed from the device so that the system may be affixed to the skin. The release liner should be made from a drug/enhancer/vehicle impermeable material, and is a disposable element, which serves only to protect the device prior to application. Typically, the release liner is formed from a material impermeable to the pharmacologically active agent and the base enhancer, and is easily stripped from the transdermal patch prior to use.

The drug-containing reservoir and skin contact adhesive may also be present in a transdermal delivery system of the invention as separate and distinct layers, with the adhesive underlying the reservoir. In such a case, the reservoir may be a polymeric matrix as described above. Alternatively, the reservoir may be comprised of a liquid or semisolid formulation contained in a closed compartment or pouch, or it may be a hydrogel reservoir, or may take some other form. As will be appreciated by those skilled in the art, hydrogels are macromolecular networks that absorb water and thus swell but do not dissolve in water. That is, hydrogels contain hydrophilic functional groups that provide for water absorption, but the hydrogels are comprised of crosslinked polymers that give rise to aqueous insolubility. Generally, then, hydrogels are comprised of crosslinked hydrophilic polymers such as a polyurethane, a polyvinyl alcohol, a polyacrylic acid, a polyoxyethylene, a polyvinylpyrrolidone, a poly(hydroxyethyl methacrylate) (poly(HEMA)), or a copolymer or mixture thereof. Particularly preferred hydrophilic polymers are copolymers of HEMA and polyvinylpyrrolidone.

Additional layers, e.g., intermediate fabric layers and/or rate-controlling membranes, may also be present in any of these drug delivery systems. Fabric layers may be used to facilitate fabrication of the device, while a rate-controlling membrane may be used to control the rate at which a component permeates out of the device. The component may be a drug, a base enhancer, an additional enhancer, or some other component contained in the drug delivery system.

A rate-controlling membrane, if present, will be included in the system on the skin side of one or more of the drug reservoirs. The material used to form such a membrane is selected so as to limit the flux of one or more components contained in the drug formulation. Representative materials useful for forming rate-controlling membranes include polyolefins such as polyethylene and polypropylene, polyamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, and the like.

Generally, the underlying surface of the transdermal device, i.e., the skin contact area, has an area in the range of about 5 to about 200 cm², preferably about 5 to about 100 cm², more preferably about 20 to about 60 cm². That area will vary, of course, with the amount of drug to be delivered and the flux of the drug through the body surface. Larger patches can be used to accommodate larger quantities of drug, while smaller patches can be used for smaller quantities of drug and/or drugs that exhibit a relatively high permeation rate.

Such drug delivery systems may be fabricated using conventional coating and laminating techniques known in the art. For example, adhesive matrix systems can be prepared by casting a fluid admixture of adhesive, drug, enhancer composition, and carrier onto the backing layer, followed by lamination of the release liner. Similarly, the adhesive mixture may be cast onto the release liner, followed by lamination of the backing layer. Alternatively, the drug reservoir may be prepared in the absence of drug or excipient, and then loaded by soaking in a drug/enhancer composition/carrier mixture. In general, transdermal systems of the invention are fabricated by solvent evaporation, film casting, melt extrusion, thin film lamination, die cutting, or the like. The basic permeation enhancer composition will generally be incorporated into the device during patch manufacture rather than subsequent to preparation of the device. Accordingly, the basic permeation enhancer composition will then convert a nonionized acidic drug to the ionized drug in salt form.

In a preferred delivery system, an adhesive overlayer that also serves as a backing for the delivery system is used to better secure the patch to the body surface. This overlayer is sized such that it extends beyond the drug reservoir so that adhesive on the overlayer comes into contact with the body surface. The overlayer is useful because the adhesive/drug reservoir layer may lose its adhesion a few hours after application due to hydration. By incorporating an adhesive overlayer, the delivery system will remain in place for the required period of time.

Other types and configurations of transdermal drug delivery systems may also be used in conjunction with the method of the present invention, as will be appreciated by those skilled in the art of transdermal drug delivery. See, for example, Ghosh, Transdermal and Topical Drug Delivery Systems (Interpharm Press, 1997), particularly Chapters 2 and 8.

As with the topically applied formulations of the invention, the drug and basic permeation enhancer composition contained within the drug reservoir(s) of these laminated systems may comprise a number of additional components. In some cases, the drug and enhancer may be delivered neat, i.e., in the absence of additional liquid. In most cases, however, the drug will be dissolved, dispersed, or suspended in a suitable pharmaceutically acceptable vehicle, typically a solvent or gel. Other components that may be present include preservatives, stabilizers, surfactants, solubilizers, additional enhancers, and the like.

It is to be understood that while the invention has been described in conjunction with the preferred specific embodiments thereof, the foregoing description is intended to illustrate and not limit the scope of the invention. Other aspects, advantages, and modifications will be apparent to those skilled in the art to which the invention pertains. Furthermore, the practice of the present invention will employ, unless otherwise indicated, conventional techniques of drug formulation, particularly topical and transdermal drug formulation, which are within the skill of the art. Such techniques are fully explained in the literature. See Remington: The Science and Practice of Pharmacy, cited supra, as well as Goodman & Gilman's The Pharmacological Basis of Therapeutics, 10^{th} Ed. (2001).

All patents, patent publications, articles, and other references cited in this application are hereby incorporated by reference in their entireties.

The following examples are put forth so as to provide those of ordinary skill in the art with a complete disclosure and description of how to practice the methods as well as make and use the compositions of the invention, and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C., and pressure is at or near atmospheric.

### Example 1

An in vitro skin permeation study was conducted using four solutions of meloxicam each containing sodium hydroxide and a nitrogenous base. The components used to prepare each formulation are listed in Table 1, along with the actual weight of each component and the weight percent in each formulation. Each component was added in the order listed in Table 1. The solution was placed directly on human cadaver skin as described below.

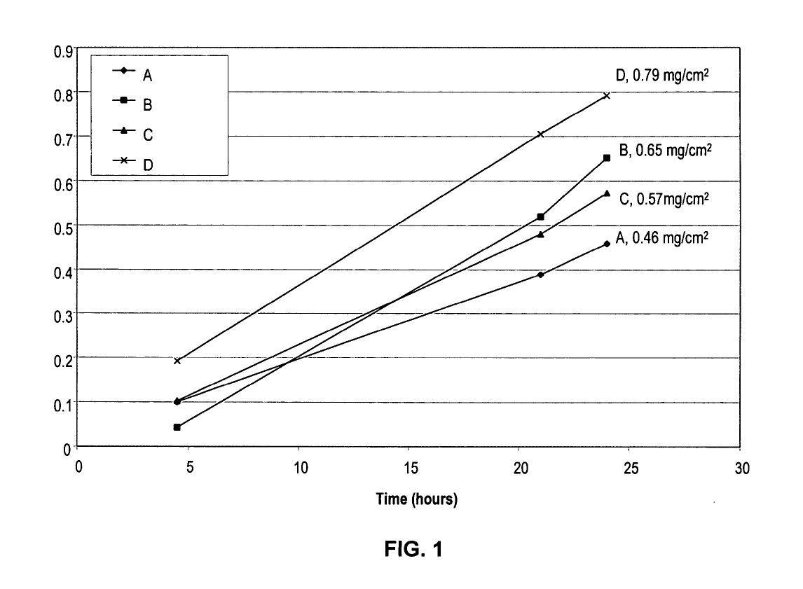

The in vitro permeation of meloxicam from formulations A, B, C, and D was evaluated using a Franz-type diffusion cells with a diffusion area of 1 cm². The volume of the receiver solution was 8 ml. Human cadaver skin was cut to a proper size and placed upon the receiver chamber of the diffusion cell with the stratum corneum facing up. The donor chamber was placed atop the stratum corneum, and the cell was clamped together. 50 μl of each meloxicam formulation was added to the stratum corneum clamped between the donor and receiver chambers of the cell. Three diffusion cells were used for each formulation. The receiver chamber was filled with a 0.5M KH₂PO₄ solution (adjusted to pH 6.5). The receiver solution and diffusion cells were maintained at 32° C. The receiver solution was completely withdrawn and replaced with fresh phosphate buffered receiver solution at each time point. The cumulative amounts of meloxicam that permeated through the human cadaver skin were calculated using the measured meloxicam concentrations in the receiver solutions, and then plotted versus time. The results are shown in FIG. 1.

The cumulative amount of meloxicam that permeated through the skin was 0.46 mg/cm² when no nitrogenous base was added. When the N-methyl glucamine was added, the cumulative amount of meloxicam that permeated through the skin was 0.65 mg/cm². When triethanolamine was added, the cumulative amount of meloxicam that permeated through the skin was 0.57 mg/cm². When the nitrogenous base urea was added, the cumulative amount of meloxicam that permeated through the skin was 0.79 mg/cm². The cumulative amount of meloxicam that permeated across human cadaver skin in 24 hours from the system containing urea was 0.79 mg/cm², which was about 1.7 times higher than when no nitrogenous base was present in the formulation.

TABLE 1
Weight and Weight Percent of Components (Based on Total Solution Weight)

| | Formulation A | Formulation B | Formulation C | Formulation D |
|---|---|---|---|---|
| Component | wt., g (wt. %) | wt., g (wt. %) | wt., g (wt. %) | wt., g (wt. %) |
| Meloxicam | 0.35 (15.5%) | 0.35 (15.5%) | 0.35 (15.5%) | 0.35 (15.5%) |
| Hexylene Glycol | 0.90 (39.8%) | 0.70 (31.0%) | 0.70 (31.0%) | 0.70 (31.0%) |
| N-Methyl | — | 0.35 (15.5%) | — | — |
| Glucamine | | | | |
| Triethanolamine | — | — | 0.35 (15.5%) | — |
| Urea | — | — | — | 0.35 (15.5%) |
| Glycerin | 0.65 (28.8%) | 0.50 (22.1%) | 0.50 (22.1%) | 0.50 (22.1%) |
| DI H₂O | 0.305 (13.5%) | 0.305 (13.5%) | 0.305 (13.5%) | 0.305 (13.5%) |
| NaOH | 0.055 (2.4%) | 0.055 (2.4%) | 0.055 (2.4%) | 0.055 (2.4%) |

### Example 2

An in vitro skin permeation study was conducted using four diclofenac sodium transdermal delivery systems. The components used to prepare each system are listed in Table 2, along with the actual weight of each component and the weight percent (based on total solution weight) in each formulation. Each formulation was coated on a release liner and dried in an oven at 65° C. for two hours to remove water and other solvents. The dried drug-in-adhesive/release liner film was laminated to a backing film, and the backing/drug-in-adhesive/release liner laminate was then cut into discs with a diameter of 9/16 inch. The theoretical weight percent weight of each component of the system, after drying (calculated assuming all volatile ingredients were completely removed during drying), is set forth in Table 3.

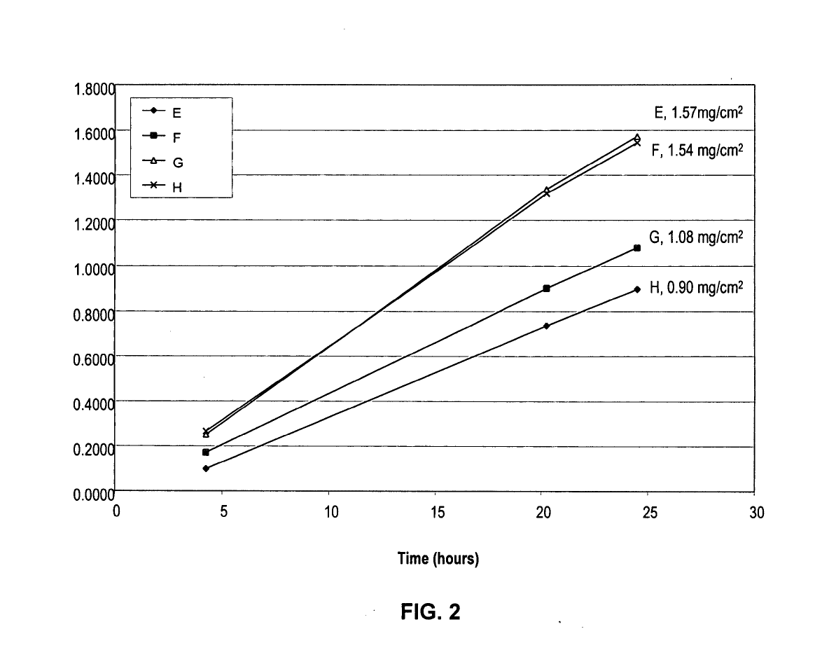

The in vitro permeation of diclofenac sodium through human cadaver skin from these discs was evaluated using Franz diffusion cells with a diffusion area of 1 cm² and a receiver solution capacity of 8 ml. Human cadaver skin was cut to a proper size and placed on a flat surface with the stratum corneum side facing up. The release liner was peeled away from the disc laminate. The backing/drug-in-adhesive film was then pressed onto the skin with the adhesive side facing the stratum corneum. The skin/adhesive/backing laminate was clamped between the donor and receiver chambers of the diffusion cell with the skin side facing the receiver solution. Three diffusion cells were used for each formulation. The receiver solution was a 0.05M KH₂PO₄ solution adjusted to pH 6.5. All cells and receiver solution were maintained at 32° C. for the 24-hour duration of the study. The entire receiver solution was collected and replaced with fresh phosphate buffered solution at each time point. The receiver solution collected was analyzed by HPLC to determine the concentration of diclofenac sodium. The cumulative amount of diclofenac sodium that permeated across the human cadaver skin was calculated using the measured diclofenac concentrations in the receiver solutions, which were plotted versus time and shown in FIG. 2.

The cumulative amount of diclofenac that permeated through the skin was 0.90 mg/cm² when no nitrogenous base was added. When the nitrogenous base triethanolamine was added, the cumulative amount of diclofenac that permeated through the skin was 1.08 mg/cm². When the N-methyl glucamine was added, the cumulative amount of diclofenac that permeated through the skin was 1.54 mg/cm². When monoethanolamine was added, the cumulative amount of diclofenac that permeated through the skin was 1.57 mg/cm². The cumulative amount of diclofenac that permeated across human cadaver skin in 24 hours from the system containing monoethanolamine was 1.57 mg/cm², which was about 1.7 times higher than when no nitrogenous base was present in the formulation.

TABLE 2
Weight and Weight Percent of Components (Based on Total Solution Weight)

| | Formulation E | Formulation F | Formulation G | Formulation H |
|---|---|---|---|---|
| Component | wt., g (wt. %) | wt., g (wt. %) | wt., g (wt. %) | wt., g (wt. %) |
| Diclofenac Sodium | 0.30 (3.3%) | 0.30 (3.3%) | 0.30 (3.3%) | 0.30 (3.3%) |
| Hexylene Glycol | 1.00 (11.0%) | 1.00 (11.0%) | 1.00 (11.0%) | 1.00 (11.0%) |
| NaOH | 0.055 (0.6%) | 0.055 (0.6%) | 0.055 (0.6%) | 0.055 (0.6%) |
| H₂O | 0.055 (0.6%) | 0.055 (0.6%) | 0.055 (0.6%) | 0.055 (0.6%) |
| Glycerin | 0.55 (6.1%) | 0.25 (2.8%) | 0.25 (2.8%) | 0.25 (2.8%) |
| Triethanolamine | — | 0.30 (3.3%) | — | — |
| Monoethanolamine | — | — | 0.30 (3.3%) | — |
| N-Methyl Glucamine | — | — | — | 0.30 (3.3%) |
| Polyvinylpolypyrrolidone | 0.60 (6.6%) | 0.60 (6.6%) | 0.60 (6.6%) | 0.60 (6.6%) |
| Polyisobutylene | 6.50 (71.7%) | 6.50 (71.7%) | 6.50 (71.7%) | 6.50 (71.7%) |
| Adhesive (30% solid) | | | | |

TABLE 3
Weight and Theoretical Weight Percent of Components in the Dried Film

| | Formulation E | Formulation F | Formulation G | Formulation H |
|---|---|---|---|---|
| Component | wt., g (wt. %) | wt., g (wt. %) | wt., g (wt. %) | wt., g (wt. %) |
| Diclofenac Sodium | 0.30 (6.7%) | 0.30 (6.7%) | 0.30 (6.7%) | 0.30 (6.7%) |
| Hexylene Glycol | 1.00 (22.4%) | 1.00 (22.4%) | 1.00 (22.4%) | 1.00 (22.4%) |
| NaOH | 0.055 (1.2%) | 0.055 (1.2%) | 0.055 (1.2%) | 0.055 (1.2%) |
| Glycerin | 0.55 (12.3%) | 0.25 (5.6%) | 0.25 (5.6%) | 0.25 (5.6%) |
| Triethanolamine | — | 0.30 (6.7%) | — | — |
| Monoethanolamine | — | — | 0.30 (6.7%) | — |
| N-Methyl Glucamine | — | — | — | 0.30 (6.7%) |
| Polyvinylpolypyrrolidone | 0.60 (13.5%) | 0.60 (13.5%) | 0.60 (13.5%) | 0.60 (13.5%) |
| Polyisobutylene | 1.95 (43.8%) | 1.95 (43.8%) | 1.95 (43.8%) | 1.95 (43.8%) |
| Adhesive (30% solid) | | | | |

### Example 3

An in vitro skin permeation study was conducted as in Example 1, using three solutions of alendronate sodium each containing sodium hydroxide and a nitrogenous base. The components used to prepare each formulation are listed in Table 4, along with the actual weight of each component and the weight percent in each formulation. Each component was added in the order listed in Table 4. The solution was placed directly on human cadaver skin as described below.

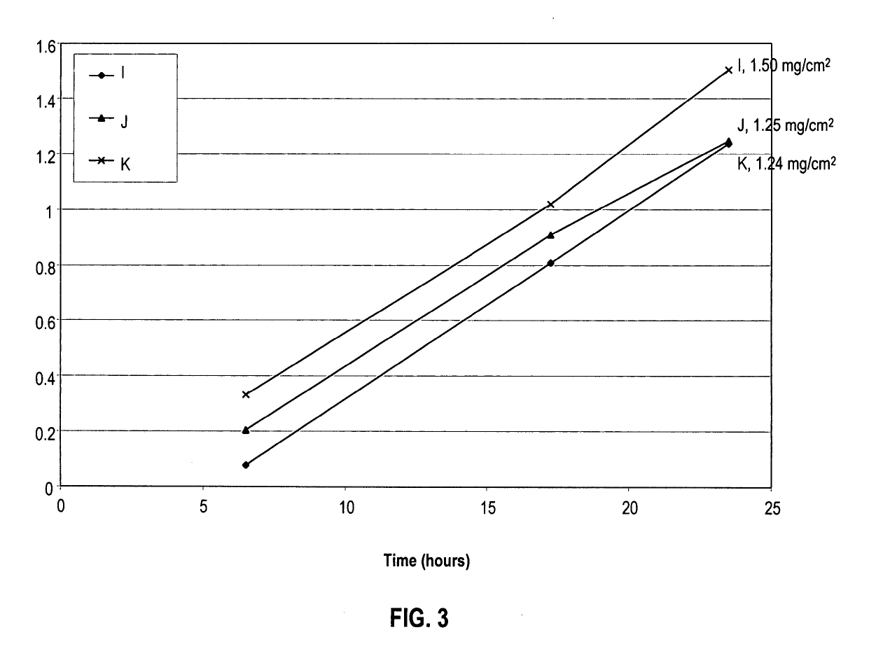

The in vitro permeation of alendronate from formulations I, J, and K was evaluated using a Franz-type diffusion cells with a diffusion area of 1 cm². The volume of the receiver solution was 8 ml. Human cadaver skin was cut to a proper size and placed upon the receiver chamber of the diffusion cell with the stratum corneum facing up. The donor chamber was placed atop the stratum corneum, and the cell was clamped together. 50 μl of each alendronate sodium solution was added to the stratum corneum clamped between the donor and receiver chambers of the cell. Three diffusion cells were used for each formulation. The receiver chamber was filled with a 0.5M KH₂PO₄ with 0.9% NaCl solution (adjusted to pH 6.5). The receiver solution and diffusion cells were maintained at 32° C. The receiver solution was completely withdrawn and replaced with fresh phosphate buffered saline receiver solution at each time point. The cumulative amount of alendronate sodium that permeated through the human cadaver skin was calculated using the measured alendronate sodium concentrations in the receiver solutions, which were plotted versus time and plotted and shown in FIG. 3.

The cumulative amount of alendronate sodium that permeated through the skin was 1.24 mg/cm² when no nitrogenous base was added. When urea was added, the cumulative amount of alendronate sodium that permeated through the skin was 1.25 mg/cm². When N-methyl glucamine and triethanolamine were added, the cumulative amount of alendronate sodium that permeated through the skin was 1.50 mg/cm². The cumulative amount of alendronate sodium that permeated across human cadaver skin in 23.5 hours from the system containing both N-methyl glucamine and triethanolamine was 1.50 mg/cm², which was about 1.2 times higher than when no nitrogenous base was present in the formulation.

TABLE 4
Weight and Weight Percent of Components (Based on Total Solution Weight)

| | Formulation I | Formulation J | Formulation K |
|---|---|---|---|
| Component | wt., g (wt. %) | wt., g (wt. %) | wt., g (wt. %) |
| Alendronate Sodium | 0.30 (11.5%) | 0.30 (11.5%) | 0.30 (11.5%) |
| NaOH | 0.15 (5.8%) | 0.15 (5.8%) | 0.15 (5.8%) |
| N-Methyl Glucamine | — | — | 0.30 (11.5%) |
| Triethanolamine | — | — | 0.30 (11.5%) |
| Urea | — | 0.30 (11.5%) | — |
| DI H₂O | 2.15 (82.7%) | 1.85 (71.2%) | 1.55 (59.6%) |

1. A method for enhancing the flux of a hydrophilic drug through a body surface, comprising:
(a) administering a therapeutically effective amount of the hydrophilic drug to a localized region of a human patient's body surface; and
(b) applying an effective flux-enhancing amount of a basic permeation enhancer composition to the localized region, the enhancer composition comprising an admixture of (i) an inorganic hydroxide and (ii) a nitrogenous base, wherein a 0.1M aqueous solution of the nitrogenous base has a pH that is about 1.0 to about 6.5 lower than the pH of a 0.1M aqueous solution of the inorganic hydroxide, and the molar ratio of the nitrogenous base to the inorganic hydroxide in the enhancer composition is in the range of about 0.5n:1 to about 20n:1, where n is the number of hydroxide ions per molecule of the inorganic hydroxide.

2. The method of claim 1, wherein a 0.1M aqueous solution of the nitrogenous base has a pH that is at least about 1.5 lower than the pH of a 0.1M aqueous solution of the inorganic hydroxide.

3. The method of claim 2, wherein the molar ratio of the nitrogenous base to the inorganic hydroxide in the composition is in the range of about 0.5n:1 to about 10n:1.

4. The method of claim 1, wherein the effective flux-enhancing amount of the composition corresponds to an amount sufficient to provide a pH within the range of about 8.0 to about 13.0 at the localized region of the body surface during administration of the drug.

5. The method of claim 4, wherein the pH is within the range of about 8.5 to about 11.5.

6. The method of claim 5, wherein the pH is within the range of about 9.5 to about 11.5.

7. The method of claim 1, wherein the inorganic hydroxide is selected from ammonium hydroxide, alkali metal hydroxides, alkaline earth metal hydroxides, and combinations thereof.

8. The method of claim 7, wherein the inorganic hydroxide is an alkali metal hydroxide.

9. The method of claim 8, wherein the alkali metal hydroxide is selected from sodium hydroxide and potassium hydroxide.

10. (canceled)

11. The method of claim 1, wherein the nitrogenous base is an amino alcohol.

12. The method of claim 1, wherein the amino alcohol is of the structural formula NR¹R²R³ wherein R¹ is hydroxy-substituted hydrocarbyl, and R² and R³ are selected from H, hydrocarbyl, and hydroxy-substituted hydrocarbyl.

13. The method of claim 12, wherein R¹ is C₁-C₁₂ alkyl substituted with 1 to 12 hydroxyl groups, and R² and R³ are selected from H, C₁-C₁₂ alkyl, and C₁-C₁₂ alkyl substituted with 1 to 12 hydroxyl groups.

14. The method of claim 13, wherein R¹ is C₁-C₆ alkyl substituted with 1 to 5 hydroxyl groups, and R² and R³ are selected from H, C₁-C₆ alkyl, and C₁-C₆ alkyl substituted with 1 to 5 hydroxyl groups.

15. (canceled)

16. (canceled)

17. The method of claim 14, wherein R¹ is —CH₂—[CH(OH)]₄—C—H₂OH, R² is CH₃, and R³ is H, such that the amino alcohol is N-methyl glucamine.

18. The method of claim 1, wherein the apparent aqueous solubility of the hydrophilic drug increases with increasing pH.

19. The method of claim 18, wherein the apparent aqueous solubility of the hydrophilic drug is greater than 2.5 mg/ml at 25° C.

20. The method of claim 19, wherein the apparent aqueous solubility of the hydrophilic drug is greater than 10 mg/ml at 25° C.

21. The method of claim 18, wherein the hydrophilic drug is an ionizable drug.

22. (canceled)

23. The method of claim 1, wherein the body surface is skin.

24. (canceled)

25. The method of claim 1, wherein the hydrophilic drug and the basic permeation enhancer composition are present in a single pharmaceutical formulation.

26. The method of claim 1, wherein the hydrophilic drug and the basic permeation enhancer composition are present in separate pharmaceutical formulations.

27. The method of claim 26, wherein (a) and (b) are carried out simultaneously.

28. The method of claim 26, wherein (a) is carried out prior to (b).

29. The method of claim 26, wherein (b) is carried out prior to (a).

30. (canceled)

31. The method of claim 25, wherein the formulation is a gel, cream, lotion, or paste.

32. The method of claim 1, wherein the hydrophilic drug is selected from analgesic agents, anesthetic agents, anti-anginal agents, antiarthritic agents, anti-arrhythmic agents, antiasthmatic agents, antibiotic agents, anticancer agents, anticholinergic agents, anticoagulants, anticonvulsants, antidepressants, antidiabetic agents, antifungal agents, antiglaucoma agents; antigout agents, antihelminthic agents, antihistamines, antihyperlipidemic agents, antihypertensive agents, antiinflammatory agents, antimalarial agents, antimigraine agents, antimuscarinic agents, antinauseants, anti-obesity agents, anti-osteoporosis agents, antipanic agents; antiparkinsonism agents, antiprotozoal agents, antipruritics, antipsychotic agents, antipyretics, antitubercular agents, antitussive agents, antiulcer agents, antiviral agents, anxiolytics, appetite suppressants, calcium channel blockers, cardiac inotropic agents, beta-blockers, bone density regulators, central nervous system stimulants, cognition enhancers, corticosteroids, decongestants, diuretics, gastrointestinal agents, genetic materials, hormonolytics, hypnotics, hypoglycemic agents, immunosuppressants, keratolytics, leukotriene inhibitors, macrolides, mitotic inhibitors, muscle relaxants, narcotic antagonists, neuroleptic agents, nicotine, parasympatholytic agents, peptides, polypeptides, proteins, saccharides, sedatives, sex hormones, sympathomimetic agents, tocolytics, tranquilizers, vasodilators, vitamins, and combinations thereof.

33. The method of claim 32, wherein the hydrophilic drug is an antiinflammatory agent.

34. The method of claim 33, wherein the antiinflammatory agent is selected from acetylsalicylic acid, alclofenac, alminoprofen, benoxaprofen, butibufen, bucloxic acid, carprofen, celecoxib, clidenac, diclofenac, diflunisal, etodolac, fenbufen, fenoprofen, fentiazic, flufenamnic acid, flufenasol, flurbiprofen, furofenac, ibufenac, ibuprofen, indomethacin, indoprofen, isoxepac, isoxicam, ketoprofen, ketorolac, meclofenamic acid, mefenamic acid, meloxicam, miroprofen, naproxen, oxaprozin, oxyphenbutazone, oxpinac, parecoxib, phenylbutazone, piclamilast, piroxicam, pirprofen, pranoprofen, rofecoxib, sudoxicam, sulindac, suprofen, tenclofenac, tiaprofenic acid, tolfenamic acid, tolmetin, tramadol, valdecoxib, zomepirac, and pharmacologically active basic addition salts thereof

35-40. (canceled)
